(19) United States
(12) Patent Application Publication
(10) Pub. No.: US 2012/0225834 A1
(43) Pub. Date: Sep. 6, 2012

(54) OCULAR IONTOPHORESIS OF CHARGED MICELLES CONTAINING BIOACTIVE AGENTS

(75) Inventors: Peyman Moslemy, Mansfield, MA (US); Hong Wang, Waltham, MA (US); Michael Patane, Andover, MA (US)

(73) Assignee: EYEGATE PHARMACEUTICALS, INC., Waltham, MA (US)

(21) Appl. No.: 13/384,518

(22) PCT Filed: Sep. 29, 2010
(86) PCT No.: PCT/US2010/050672

§ 371 Date: May 18, 2012

Related U.S. Application Data
(60) Provisional application No. 61/246,835, filed on Sep. 29, 2009.

(51) Int. Cl. A61K 31/7048; A61P 27/02; A61K 31/56

(52) U.S. Cl. 514/31; 514/178

(57) ABSTRACT

This invention is related to pharmaceutical compositions consisting of charged micelles containing bioactive agents. The invention is also related to the methods of delivery of charged micelles to the eye of a mammal by iontophoresis.

### FIELD OF THE INVENTION

This invention is related to pharmaceutical compositions consisting of charged micelles containing bioactive agents and methods of iontophoretic delivery thereof.

### BACKGROUND OF THE INVENTION

Conventional ophthalmic drug delivery methods employ systemic, topical and injection modes of administration. In particular, topical applications account for the widest use of non-invasively delivered bioactive agents for ocular disorders. This approach, however, suffers from limited efficacy due to low bioavailability and slow, inadequate and uneven drug uptake.

Because current ocular delivery methods achieve low ocular exposures, frequent applications are required and compliance issues are significant. Additionally, certain properties of molecules make them less- and/or unsuitable for iontophoresis; for example, molecules that lack a charge, water solubility, and/or stability across a range of pHs.

Pharmaceuticals require customized formulations for iontophoresis. These alterations maximize dosing effectiveness, improve the safety and manage commercial challenges. A need remains in the art for a composition and method for prevention and treatment of ocular conditions.

### SUMMARY OF THE INVENTION

The present technology relates to charged micelles containing bioactive agents suited for ocular iontophoresis. These compositions may be suitable for treating a variety of ocular disorders. The present technology also relates to method for prophylactic and therapeutic treatment of a subject having an ocular disease or condition.

In one aspect, the present technology provides a pharmaceutical composition comprising one or multiple populations of normal-phase micelles comprising a charged surfactant and a bioactive agent, wherein each population of micelles is differentiated with respect to the quantity of micelles, and type and or quantity of the said ingredients.

In one embodiment, the surfactant is a cationic surfactant.

In one embodiment, the cationic surfactant is a quaternary ammonium cationic surfactant (QACS).

In one embodiment, the quaternary ammonium cation of said surfactant is selected from the group consisting of alkylammonium, benzylammonium, pyridinium, and imidazolium ions.

In one embodiment, the QACS is selected from the group consisting of alkyltrimethylammonium halide, alkyldimethylammonium halide, alkylmethylammonium halide, alkylethyldimethylammonium halide, alkyldimethylbenzylammonium halide, alkylpyridinium halide, and alkylimidazolium halide, or a mixture of two or more thereof.

In one embodiment, the QACS is selected from the group consisting of decyltrimethylammonium halide, lauryltrimethylammonium halide, cetyltrimethylammonium halide, cetylethyldimethylammonium halide, octadecyltrimethylammonium halide, didodecyldimethylammonium halide, ditetradecyldimethylammonium halide, dioctadecyldimethylammonium halide, and trioctadecylmethylammonium halide, or a mixture of two or more thereof.

In one embodiment, the quaternary ammonium ion is Dodecyldimethylbenzylammonium halide (benzododecinium halide).

In one embodiment, the quaternary ammonium ion is cetylpyridinium halide.

In one embodiment, the surfactant is an anionic surfactant.

In one embodiment, the anionic surfactant is selected from the group consisting of sodium oleate, sodium lauryl sulfate, sodium cetyl sulfate, sodium stearyl sulfate, alkylbenzene sulfonic acid salts such as sodium alkylbenzene sulfonate, dialkyl sulfosuccinic acid salts, dioctylester of sulphosuccinic acid salts; and sodium salt of alkylated aryl polyether sulfate.

In one embodiment, the bioactive agent has an aqueous solubility of greater than 1000 mg/mL.

In one embodiment, the bioactive agent has an aqueous solubility of 100 to 1000 mg/mL.

In one embodiment, the bioactive agent has an aqueous solubility of 33 to 100 mg/mL.

In one embodiment, the bioactive agent has an aqueous solubility of 10 to 33 mg/mL.

In one embodiment, the bioactive agent has an aqueous solubility of 1 to 10 mg/mL.

In one embodiment, the bioactive agent has an aqueous solubility of 0.1 to 1 mg/mL.

In one embodiment, the bioactive agent has an aqueous solubility of less than 0.1 mg/mL.

In one embodiment, the bioactive agent comprises from about 0.5 to about 30 percent by weight of the micelle composition.

In one embodiment, the bioactive agent comprises from about 1 to about 20 percent by weight of the micelle composition.

In one embodiment, the bioactive agent comprises from about 2 to about 10 percent by weight of the micelle composition.

In one embodiment, the pharmaceutical composition contains a bioactive agent suitable for ocular therapy of a mammal.

In one embodiment, the pharmaceutical composition is suitable for delivery to the eye of a mammal by iontophoresis.

In one embodiment, the method of delivery to the eye is transscleral iontophoresis.

In one embodiment, the method of delivery to the eye is transcorneal iontophoresis.

In one embodiment, the pharmaceutical composition is suitable for delivery to the eye of a mammal by topical instillation.

In exemplary embodiments, a bioactive agent is a synthetic or a natural compound which demonstrates a biological effect when introduced into a living creature. Such agents may include diagnostic and therapeutic agents including both large and small molecules intended for the treatment of acute or chronic conditions.

In some embodiments, a bioactive agent is an ophthalmic drug including, but not limited to, e.g., small molecules, and biologics such as peptides, oligopeptides, proteins and antibodies, antibodies fragments, aptamers, oligonucleotides, and small interfering RNAs.

In some embodiments, a bioactive agent is an antibacterial agent, antifungal agent, antiviral agent, antiglaucomatous agent, anti-histamine, anti-proliferative agent, anti-inflammatory agent, non-steroidal anti-inflammatory drug, anti-VEGF (vascular endothelial growth factor) agent, anti-cancerous agent, decongestant, anti-diabetic agent, immunomodulator, and/or a drug for central nervous and movement disorders.

In another embodiment, the pharmaceutical composition is suitable for delivery to the eye of a mammal by an injection method, including periocular injection, retrobulbar injection, peribulbar injection, subconjunctival injection, and intravitreal injection.

In one embodiment, the micelles are characterized by a positive surface charge or zeta potential of from about (+) 20 mV to about (+) 100 mV.

In one embodiment, the micelles are characterized by a positive surface charge or zeta potential of from about (+) 40 mV to about (+) 80 mV.

In one embodiment, the micelles are characterized by a negative surface charge or zeta potential of from about (−) 20 mV to about (−) 100 mV.

In one embodiment, the micelles are characterized by a negative surface charge or zeta potential of from about (−) 40 mV to about (−) 80 mV.

In one embodiment, the micelles are characterized by a diameter of from about 1 nm to about 100 nm.

In one embodiment, the micelles are characterized by a diameter of from about 10 nm to about 50 nm.

In another embodiment, the pharmaceutical composition also contains inactive ingredients such as buffering agents, osmotic agents, penetration or absorption enhancers, chelants, antioxidants, preservatives, pH adjusting agents, viscosity modifying agents, lubricating agents, non-ionic surfactants, and cryopreservative agents.

In another aspect, the present technology also relates to a method for preventing or treating ocular conditions, comprising administering an effective amount of charged micelles comprising a charged surfactant and a bioactive agent by ocular iontophoresis.

### BRIEF DESCRIPTION OF THE DRAWINGS

FIG. 1 is a graph showing dexamethasone concentration as a function of surfactant concentration. Dissolved concentration of dexamethasone in aqueous solutions of quaternary ammonium cationic surfactants; CTAB: cetyltrimethylammonium bromide, TMAB: ditetradecyldimethylammonium bromide, and OMAC, dioctadecyldimethylammonium chloride.

FIG. 2 is a graph showing natamycin concentration as a function of surfactant concentration. Dissolved concentration of natamycin in aqueous solutions of quaternary ammonium cationic surfactants; CTAB: cetyltrimethylammonium bromide, TMAB: ditetradecyldimethylammonium bromide, and OMAC, dioctadecyldimethylammonium chloride.

### DETAILED DESCRIPTION

### I. Charged Micelle Compositions of the Present Technology.

The present technology provides charged micelle compositions containing bioactive agents suited for ocular iontophoresis. Advantages of compositions of present technology to prior art include, but are not limited to: (1) the use of ionic surfactants (described below) to create highly charged micelles capable of enclosing and electro-mobilizing a bioactive agent; (2) the use of ionic surfactants to create highly charged micelles capable of solubilizing, enclosing and electro-mobilizing a poorly soluble bioactive agent; (3) the use of ionic surfactants to create highly charged micelles capable of solubilizing, enclosing and electro-mobilizing a poorly soluble and un-charged bioactive agent; (4) the employment of drug carrying charged micelles for treating various diseases of the eye by iontophoresis.

A. General

A micelle is an aggregate of surfactant molecules dispersed in a liquid colloid. A colloid is a type of chemical mixture where one substance is dispersed evenly throughout another. A typical micelle in aqueous solution forms an aggregate with the hydrophilic head regions in contact with surrounding solvent, sequestering the hydrophobic single tail regions in the micelle center. This type of micelle is known as a normal phase micelle (oil-in-water micelle). Inverse micelles have the head groups at the centre with the tails extending out (water-in-oil micelle). Micelles only form when the concentration of surfactant is greater than the critical micelle concentration (CMC), and the temperature of the system is greater than the critical micelle temperature, or Krafft temperature. Micelles are approximately spherical in shape. Other phases, including shapes such as ellipsoids, cylinders, and bilayers are also possible. The shape and size of a micelle is a function of the molecular geometry of its surfactant molecules and solution conditions such as surfactant concentration, temperature, pH, and ionic strength.

Individual surfactant molecules that are in the aqueous solution but are not part of a micelle are called monomers. In water, the hydrophilic heads of surfactant molecules are always in contact with the solvent, regardless of whether the surfactants exist as monomers or as part of a micelle. However, the lipophilic tails of surfactant molecules have less contact with water when they are part of a micelle—this being the basis for the energetic drive for micelle formation. In a micelle, the hydrophobic tails of several surfactant molecules assemble into an oil-like core the most stable form of which has no contact with water. By contrast, surfactant monomers are surrounded by water molecules that create a cage of molecules connected by hydrogen bonds.

Micelles composed of ionic surfactants have an electrostatic attraction to the ions that surround them in the solution, the latter known as counterions. Although the closest counterions partially mask a charged micelle, the effects of micelle charge affect the structure of the surrounding solvent at appreciable distances from the micelle. Ionic micelles influence many properties of the mixture, including its electrical conductivity. Environmental variables such as pH, temperature, and ionic strength in a colloid containing micelles can decrease the strength of electrostatic interactions and lead to the formation of larger ionic micelles.

B. Charge Characteristics of Compositions of the Present Technology

The relatively high positive charge (zeta potential) of micelles of this invention which in turn contributes to their physical stability in a suspension form makes them suitable for topical delivery to ocular surface tissues, which are negatively charged at physiological pH (>4). Yet the positive charge of micelles enables their electrophoretic mobility in anodal iontophoresis applications.

The surface charge of micelles is influenced by the composition of micelles and can be varied mainly by varying the surfactant type and concentration, and buffering agent(s) type and concentration (and any other ionic components of the composition). In general, the surface charge of micelles is also influenced by the chemistry (pH and ionic strength) of their surrounding environment.

In general, the surface charge of colloidal particles is represented by zeta potential. Zeta potential can be used to predict the electrophoretic mobility and physical stability of charged micelles in a colloid. In order to prevent aggregation and successive size growth of micelles, it is necessary to confer repulsive forces to the micellae by electrostatic or charge stabilization. Electrostatic or charge stabilization has the benefits of stabilizing a colloid by simply altering the concentration of ions surrounding the micelles. Ionization of hydrophilic heads of ionic surfactants can help the creation of a stable micellar formulation. In the pharmaceutical compositions of the present technology, positively-charged micellae containing bioactive agents are produced by employing one or more of cationic surfactants while negatively-charged micellae containing bioactive agents are generated by using one or more of anionic surfactants.

The interaction of colloidal particles in polar liquids such as water is not governed by the electrical potential at the surface of the micelle, but by the effective potential of the micelle and its associated ions. To utilize electrostatic control of dispersions, it is the zeta potential of the micelle that must be measured. Charged micelles will attract ions of opposite charge in the dispersant. Ions close to the surface are strongly bound; those further away form a more diffuse region. Within this region is a notional boundary, known as the slipping plane, within which the micelle and ions act as a single entity. The potential at the slipping plane is known as the zeta potential. It has long been recognized that the zeta potential is a very good index of the magnitude of the interaction between colloidal particles and their electrophoretic mobility. Measurements of zeta potential are commonly used to assess the stability of colloidal systems. The zeta potential measured in a particular system is dependent on the chemistry of the surface, and also of the way it interacts with its surrounding environment. Therefore zeta potential must always be studied in a well defined environment (i.e. known pH and ionic strength).

An important consequence of the existence of electrical charges on the surface of micelles is that they interact with an applied electric field. These effects are collectively defined as electrokinetic effects. If the motion is induced in a particle suspended in a liquid under the influence of an applied electric field, it is more specifically named electrophoresis. When an electric field is applied across an electrolyte, charged micelles suspended in the electrolyte are attracted towards the electrode of opposite charge. Viscous forces acting on the micelles tend to oppose this movement. When equilibrium is reached between these two opposing forces, the micelles move with constant velocity. The velocity is dependent on the strength of electric field or voltage gradient, the dielectric constant of the medium, the viscosity of the medium and the zeta potential. The velocity of a particle in a unit electric field is referred to as its electrophoretic mobility. Zeta potential is related to the electrophoretic mobility by the Henry's equation:

$${U}_{e} = \frac{2 {ɛɛ}_{0} ϛ}{3 η} f ( κ a ) \qquad ( I )$$

where Uₑ is electrophoretic mobility, c is the dielectric constant of the dispersion medium, ε₀ is the permittivity of free space, ζ is the zeta potential, η is the dynamic viscosity of the dispersion medium, and R(κa) is Henry's function. For small particles (i.e. <200 nm) in aqueous media and moderate electrolyte concentration f(κa) is 1.0, and this is referred to as the Huckel approximation.

When surfactants are present above the CMC, they can act as emulsifiers that will allow a compound normally insoluble (in the solvent being used) to dissolve. This occurs because the insoluble species can be incorporated into the micelle core, which is itself solubilized in the bulk solvent by virtue of the head groups' favorable interactions with solvent species.

The combination of solubilization capacity of micelles and electrophoretic mobility of a colloid containing ionic micelles provides an opportunity for delivery of bioactive agents by iontophoresis of charged micelles.

In one embodiment of the invention, the pharmaceutical compositions are formulated for ophthalmic administration.

The pharmaceutical compositions of this invention may include inactive ingredients such as buffering agents, osmotic agents, penetration or absorption enhancers, chelants, antioxidants, preservatives, viscosity modifying agents, lubricating agents, non-ionic surfactants, and cryopreservative agents.

C. Cationic Surfactants

The micelles of this present technology may be formulated into pharmaceutical compositions with various hydrophilic or hydrophobic active ingredients. In one embodiment of the invention, the cationic surfactant is a quaternary ammonium cationic surfactant (QACS). A QACS is a salt of a nitrogenous cation in which a central nitrogen atom is bonded to four organic radicals and an anion (X), of general formula R₄N⁺X⁻ which exhibits surface active properties. In a QACS generally at least one of the R groups is a long-chain (greater than 6 carbon atoms) alkyl or aryl group. Representative quaternary ammonium surfactants include, but are not limited to, those of the alkylammonium, benzalkonium, and pyridinium families. More specifically, the QACS are selected from alkyltrimethylammonium salts, alkyldimethylammonium salts, alkylmethylammonium salts, alkyldimethylbenzylammonium salts, alkylpyridinium, and alkylimidazolium salts. An exemplary list of alkylammonium surfactants is shown in Table 1.

TABLE 1
Quaternary alkylammonium surfactants

| | | Compendial | | | |
|---|---|---|---|---|---|
| Compound | Structure | Name | Linear Formula | MW | CAS # |
| Decyltrimethylammonium bromide | 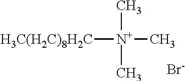 | DTAB | CH₃(CH₂)₉N(CH₃)₃(Br) | 280.29 | 2082-84-0 |
| Dodecyltrimethylammonium bromide, Lauryltrimethyl- ammonium bromide | 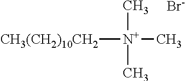 | LTAB | CH₃(CH₂)₁₁N(CH₃)₃Br | 308.34 | 1119-94-4 |
| Cetyltrimethylammonium bromide, Hexadecyltrimethyl- ammonium bromide | 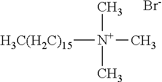 | CTAB | CH₃(CH₂)₁₅N(Br)(CH₃)₃ | 364.45 | 57-09-0 |
| Octadecyltrimethylammonium bromide | 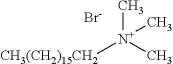 | OTAB | CH₃(CH₂)₁₇N(Br)(CH₃)₃ | 392.5 | 1120-02-1 |
| Didodecyldimethylammonium bromide | 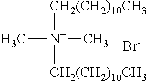 | DMAB | [CH₃(CH₂)₁₁]₂N(CH₃)₂(Br) | 462.63 | 3282-73-3 |
| Ditetradecyldimethylammonium bromide | 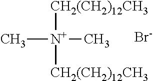 | TMAB | [CH₃(CH₂)₁₃]₂N(Br)(CH₃)₂ | 518.74 | 68105-02-2 |
| Dioctadecyldimethylammonium chloride | 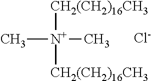 | OMAC | [CH₃(CH₂)₁₇]₂N(Cl)(CH₃)₂ | 586.5 | 107-64-2 |
| Dioctadecyldimethylammonium bromide, Distearyldimethyl- ammonium bromide | 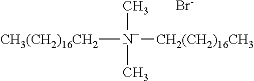 | DDAB | [CH₃(CH₂)₁₇]₂N(Br)(CH₃)₂ | 630.95 | 3700-67-2 |
| Trioctadecylmethylammonium bromide | 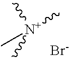 | OMAB | [CH₃(CH₂)₁₇]₃N(Br)CH₃ | 869.4 | 18262-86-7 |

In selected embodiments of the invention, the QACS is selected from the group consisting of alkyltrimethylammonium halide, alkyldimethylammonium halide, alkylmethylammonium halide, alkylethyldimethylammonium halide, alkyldimethylbenzylammonium halide, alkylpyridinium halide, and alkylimidazolium halide.

In one embodiment of the invention, the QACS is selected from decyltrimethylammonium halide, lauryltrimethylammonium halide, cetyltrimethylammonium halide, cetylethyldimethylammonium halide, octadecyltrimethylammonium halide, didodecyldimethylammonium halide, ditetradecyldimethylammonium halide, dioctadecyldimethylammonium halide, dioctadecyldimethylammonium halide, or a mixture of two or more thereof.

D. Anionic Surfactants

In one embodiment of the invention, the anionic surfactant is selected from the group consisting of sodium oleate, sodium lauryl sulfate, sodium cetyl sulfate, sodium stearyl sulfate, alkylbenzene sulfonic acid salts such as sodium alkylbenzene sulfonate, dialkyl sulfosuccinic acid salts, dioctylester of sulphosuccinic acid salts; and sodium salt of alkylated aryl polyether sulfate.

### Bioactive Agents Useful in the Compositions of the Present Technology

A bioactive agent is a synthetic or a natural compound which demonstrates a biological effect when introduced into a living creature. Such agents may include diagnostic and therapeutic agents including both large and small molecules intended for the treatment of acute or chronic conditions.

Therapeutic compounds useful in the compositions of the present technology include, but are not limited to, e.g., ophthalmic drugs including, but not limited to, e.g., small molecules, and biologics such as peptides, oligopeptides, proteins and antibodies, antibodies fragments, aptamers, oligonucleotides, and small interfering RNAs. Exemplary molecules belong to such therapeutics classes as antibacterials, antifungals, antivirals, antiglaucomatous agents, anti-histamines, anti-proliferative agents, anti-inflammatory agents, non-steroidal anti-inflammatory drugs, anti-VEGF (vascular endothelial growth factor) agents, anti-cancerous agents, decongestants, anti-diabetic agents, immunomodulators, and drugs for central nervous and movement disorders.

In one embodiment, the bioactive agent has an aqueous solubility of greater than 1000 mg/mL.

In one embodiment, the bioactive agent has an aqueous solubility of 100 to 1000 mg/mL.

In one embodiment, the bioactive agent has an aqueous solubility of 33 to 100 mg/mL.

In one embodiment, the bioactive agent has an aqueous solubility of 10 to 33 mg/mL.

In one embodiment, the bioactive agent has an aqueous solubility of 1 to 10 mg/mL.

In one embodiment, the bioactive agent has an aqueous solubility of 0.1 to 1 mg/mL.

In one embodiment, the bioactive agent has an aqueous solubility of less than 0.1 mg/mL.

In one embodiment, the bioactive agent comprises from about 0.5 to about 30 percent by weight of the micelle composition.

In one embodiment, the bioactive agent comprises from about 1 to about 20 percent by weight of the micelle composition.

In one embodiment, the bioactive agent comprises from about 2 to about 10 percent by weight of the micelle composition.

E. Particle Size of the Compositions of the Present Technology

In one embodiment, the micelles are characterized by a diameter of from about 1 nm to about 100 nm.

In another embodiment, the micelles are characterized by a diameter of from about 10 nm to about 50 nm.

Inactive Ingredients Useful in the Compositions of the Present Technology

### A. Buffering Agents

In one embodiment of the invention, optionally at least one buffering agent is utilized to control the pH of formulation that otherwise may change as a result of chemical or electrochemical interactions during use or storage of the formulation. In one embodiment, the buffer composition comprises an amino acid or a combination of amino acids with cationic behavior. In another embodiment, mixtures of a cationic amino acid buffer and an anionic acid buffer may also be used. Cationic amino acids useful in the compositions of the present technology include, but are not limited to, e.g., arginine, aspartic acid, cycteine, glutamic acid, histidine, lysine, and tyrosine. Anionic acids useful in the compositions of the present technology include, but are not limited to, e.g.,acetic acid, adipic acid, aspartic acid, benzoic acid, citric acid, ethylenediamine tetracetic acid, formic acid, fumaric acid, glutamic acid, glutaric acid, maleic acid, malic acid, malonic acid, phosphoric acid, and succinic acid.

In one embodiment, the buffer composition comprises an amino acid or a combination of amino acids with anionic behavior. In one embodiment, mixtures of an anionic amino acid buffer and an anionic acid buffer and a cationic base or cationic amino acid buffer may also be used. Anionic amino acids useful in the compositions of the present technology include, but are not limited to, e.g., cycteine, histidine, and tyrosine. Anionic acid buffers useful in the compositions of the present technology include, but are not limited to, e.g., acetic acid, adipic acid, benzoic acid, carbonic acid, citric acid, ehtylenediamine tetracetic acid, fumaric acid, glutamic acid, lactic acid, maleic acid, malic acid, malonic acid, phosphoric acid, tartaric acid, and succinic acid. Cationic bases and amino acids useful in the compositions of the present technology include, but are not limited to, e.g., arginine, histidine, imidazole, lysine, triethanolamine, and tromethamine. In one embodiment, buffering agents include zwitterions. Zwitterions useful in the compositions of the present technology include, but are not limited to, e.g., N-2(2-acetamido)-2-aminoethane sulfonic acid (ACES), N-2-acetamido iminodiacetic acid (ADA), N,N-bis(2-hydroxyethyl)-2-aminoethane sulfonic acid (BES), 2-[Bis-(2-hydroxyethyl)-amino]-2-hydroxymethyl-propane-1,3-diol (Bis-Tris), 3-cyclohexylamino-1-propane sulfonic acid (CAPS), 2-cyclohexylamino-1-ethane sulfonic acid (CHES), N,N-bis(2-hydroxyethyl)-3-amino-2-hydroxypropane sulfonic acid (DIPSO), 4-(2-hydroxyethyl)-1-piperazine propane sulfonic acid (EPPS), N-2-hydroxyethylpiperazine-N′-2-ethane sulfonic acid (HEPES), 2-(N-morpholino)-ethane sulfonic acid (MES), 4-(N-morpholino)-butane sulfonic acid (MOBS), 2-(N-morpholino)-propane sulfonic acid (MOPS), 3-morpholino-2-hydroxypropanesulfonic acid (MOPSO), 1,4-piperazine-bis-(ethane sulfonic acid) (PIPES), piperazine-N,N′-bis(2-hydroxypropane sulfonic acid) (POPSO), N-tris(hydroxymethyl)methyl-2-aminopropane sulfonic acid (TAPS), N-[tris(hydroxymethyl)methyl]-3-amino-2-hydroxypropane sulfonic acid (TAPSO), N-tris(hydroxymethyl) methyl-2-aminoethane sulfonic acid (TES), and 2-Amino-2-hydroxymethyl-propane-1,3-diol (Tris).

In one embodiment, buffering agents include a polymer or a combination of polymers with anionic or cationic behavior. The polymeric buffer may be any polymer which ionizes at a given pH by consuming hydrogen ions or hydroxyl ions and maintains the pH of the micelle composition within a desired range. Anionic polymers useful in the compositions of the present technology include, but are not limited to, e.g., poly(acrylic acid), poly(acrylic acid) crosslinked with polyalkenyl ethers or divinyl glycol, poly(methacrylic acid), styrene/maleic anhydride copolymers, methyl vinyl ether/maleic anhydride copolymers, poly(vinyl acetate phthalate), cellulose acetate phthalate, cellulose acetate trimellitate, hydroxypropyl methylcellulose phthalate, hydroxypropyl methylcellulose acetate succinate, ethyl acrylate/methacrylic acid copolymers, methyl methacrylate/methacrylic acid copolymers, and alginic acid. The cationic polymer is selected from a group consisting of polyvinylpyridine, methyl methacrylate/ butyl methacrylate/dimethylaminoethyl methacrylate terpolymers, vinylpyrrolidone/quatemized dimethyl aminoethyl methacrylate copolymers, vinylcaprolactam/vinylpyrrolidone/dimethyl aminoethyl methacrylate terpolymers, and chitosan. In yet another embodiment, the buffer composition is a crosslinked polymer or a combination of polymers with anionic or cationic behavior. In one embodiment, the polymeric buffer is an ion exchange resin, e.g., methacrylic acid/divinylbenzene copolymers and styrene/divinylbenzene copolymers. Methacrylic acid/divinylbenzene copolymers have weak acid (carboxyl group) functionality and are available in hydrogen or potassium form. Styrene/divinylbenzene polymers have either strong acid (sulfonate group) or strong base (tertiary amine group) functionality. The former resins are available in hydrogen, sodium or calcium form and while the latter resins are available in chloride form.

In one embodiment, the buffer composition is a crosslinked polymer or a combination of polymers with zwitterionic behavior. Zwitterionic polymers useful in the compositions of the present technology include, but are not limited to, e.g., poly(2-acrylamido-2-methyl-1-propane sulfonic acid) hydrogels (generally referred to as PolyAMPS), PolyAMPS/hyaluronic acid interpenetrating polymer network (IPN) hydrogels, cross-linked copolymers of AMPS and 2-hydroxyethyl methacrylate (HEMA), cross-linked copolymers of AMPS and 2-dimethylamino ethyl methacrylate (DMAEMA), and cross-linked copolymers of AMPS and acrylic acid.

Buffering agents useful in the compositions of the present technology include, but are not limited to, e.g., phosphate, citrate, or acetate buffers or combinations thereof.

### B. Osmotic Agents

In one embodiment, the formulations of the invention optionally contain at least one osmotic agent (or tonicity adjusting agent) sufficient to render the composition acceptable for administration to a mammal. Exemplary osmotic agents useful in the compositions of the present technology include, but are not limited to, e.g., sodium chloride, sodium borate, sodium acetate, sodium phosphates, sodium sulfate, potassium sulfate, calcium sulfate, magnesium sulfate, sodium hydroxide, and hydrochloric acid, mannitol, sorbitol, glucose, sucrose, lactulose, trehalose, and glycerol. Polyols, such as erythritol components, xylitol components, inositol components, and the like and mixtures thereof, are effective tonicity/osmotic agents, and may be included, alone or in combination with glycerol and/or other compatible solute agents, in the invention compositions. Other non-ionic tonicity adjusting agents useful in the compositions of the present technology include, but are not limited to, e.g., polyethylene glycols (PEG), polypropylene glycols (PPG) and mixtures thereof.

### C. Penetration Enhancers

Compositions of the invention optionally include one or more agents to enhance the body tissue penetration or absorption of micelles. For instance, the epithelium is the main barrier to drug penetration through the cornea. It is possible to enhance the penetration of drugs through the epithelium by promoting drug partition into the epithelium, thereby enhancing the overall absorption of drugs applied to the eye. The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers are required to be pharmacologically inert and chemically stable, to have a high degree of potency in terms of both specific activity and reversible effects on cornea permeability, and to be both nonirritating and nonsensitizing. The penetration enhancers and any metabolites thereof must also be non-toxic to ophthalmic tissues.

Penetration enhancers useful in the compositions of the present technology include, but are not limited to, e.g., surfactants including bile acids including deoxycholic acid, taurocholic acid, taurodeoxycholic acid, and the like; bile salts such as sodium cholate and sodium glycocholate; fatty acids such as capric acid; preservatives such as benzalkonium chloride, chlorhexidine digluconate, parabens such as methylparaben and propylparaben, chlorobuthanol, and so on; chelating agents such as ethylenediamine tetraacetic acid (EDTA) and its sodium salts; polyoxyethylene sorbitan fatty acid esters such as polyoxyethylene sorbitan monolaurate (polysorbate 20, Tween® 20); polyoxyethylene lauryl ethers such as polyoxyethylene (23) lauryl ether (Brij 35); and other compounds such as dimethyl sulfoxide (DMSO), 1-dodecylazayl-cycloheptan-2-one (Azone), hexamethylene lauramide, decylmethylsulfoxide, decamethonium bromide, saponin, and sodium fusidate. A complete list of the above penetration enhancers is provided by Sasaki et al. In: Critical Reviews in Therapeutic Drug Carrier Systems, 16(1):85-146 (1999).

Other penetration enhancers useful in the compositions of the present technology include, but are not limited to, e.g.,saccharide surfactants, such as dodecylmaltoside (DDM) and monoacyl phosphoglycerides such as lysophosphatidylcholine. The saccharide surfactants and monoacyl phosphoglycerides which may be utilized as penetration enhancers in the present invention are known compounds. The use of such compounds to enhance the penetration of ophthalmic drugs is described in the U.S. Pat. No. 5,221,696 and the U.S. Pat. No. 5,369,095, respectively.

### D. Chelants

In one aspect, the compositions of the invention may contain at least one chelating agent selected from the group consisting of sodium citrate and EDTA and its sodium salts. A chelant, as used herein, chelates metal ions which may catalyze the degradation of the micelle encapsulated drug.

### E. Antioxidants

In one embodiment, the compositions of the invention may contain at least one antioxidant. Antioxidants useful in the compositions of the present technology include, but are not limited to, e.g., alpha tocopherol (Vitamin E); cysteine; taurine; citric acid, ascorbic acid, ascorbyl palmitate, EDTA and its sodium salts; sodium bisulfite, and sodium metabisulfite. An antioxidant, as used herein, prevents or reduces the degradation of a drug which could otherwise degrade through oxidative pathways.

### F. Preservatives

In one embodiment of the invention, the formulations may contain at least one preservative. A preservative, as used herein, is an additive which inhibits microbial growth and or kills microorganisms which inadvertently contaminate a pharmaceutical composition upon exposure to the surroundings. Preservatives useful in the compositions of the present technology include, but are not limited to, e.g., hydrophobic or non-charged preservatives, anionic preservatives, and cationic preservatives. A preservative enhancing agent, as used herein, refers to an additive which increases the preservative effectiveness of a preservative, or the preservative effectiveness of a preserved formulation, but which would not typically be used solely to preserve a pharmaceutical composition.

Cationic preservatives useful in the compositions of the present technology include, but are not limited to, e.g., polymyxin B sulfate, quaternary ammonium compounds, poly(quaternary ammonium) compounds, p-hydroxybenzoic acid esters, benzalkonium chloride, benzoxonium chloride, cetylpridinium chloride, benzethonium chloride, cetyltrimethyl ammonium bromide, chlorhexidine, poly(hexamethylene biguanide), and mixtures thereof.

Anionic preservatives useful in the compositions of the present technology include, but are not limited to, e.g., sorbic acid; 1-octane sulfonic acid (monosodium salt); 9-octadecenoic acid (sulfonated); ciprofloxacin; dodecyl diphenyloxide-disulfonic acid; ammonium, potassium, or sodium salts of dodecyl benzene sulfonic acid; sodium salts of fatty acids or tall oil; naphthalene sulfonic acid; sodium salts of sulfonated oleic acid; organic mercurials such as thimerosal (sodium ethylmercurithiosalicylate); thimerfonate sodium (sodium p-ethylmercurithiophenylsulfonate).

Hydrophobic or non-ionic preservatives useful in the compositions of the present technology include, but are not limited to, e.g., 2,3-dichloro-1,4-naphthoquinone; 3-methyl-4-chlorophenol; 8-hydroxyquinoline and derivatives thereof benzyl alcohol; phenethyl alcohol; bis(hydroxyphenyl)alkanes; bisphenols; chlorobutanol; chloroxylenol; dichlorophen[2,2′-methylene-bis(4-chlorophenol)]; ortho-alkyl derivatives of para-bromophenol and para-chlorophenol; oxyquinoline; para-alkyl derivatives of ortho-chlorophenol and ortho-bromophenol; pentachlorophenyl laurate; phenolic derivatives such as 2-phenylphenol, 2-benzyl-4-chlorophenol, 2-cyclopentyl-4-chlorophenol, 4-t-amylphenol, 4-t-butylphenol, and 4and 6-chloro-2-pentylphenol; phenoxy fatty acid polyester; phenoxyethanol; methylparaben, propylparaben, and butylparaben.

### G. pH Adjusting Agents

In one embodiment, the formulations of the invention may contain at least one pH adjusting agent. pH adjusting agents useful in the compositions of the present technology include, but are not limited to, e.g., hydrochloric acid, citric acid, phosphoric acid, acetic acid, tartaric acid, sodium hydroxide, potassium hydroxide, sodium carbonate and sodium bicarbonate.

### H. Viscosity Modifying Agents

In one embodiment, the formulations of the invention may contain at least one viscosity modifying agent. Viscosity modifying agents useful in the compositions of the present technology include, but are not limited to, e.g., cellulose derivatives such as hydroxymethylcellulose, hydroxyethylcellulose, hydroxypropylcellulose, hydroxypropylmethylcellulose, methylcellulose, and carboxymethylcellulose; poly(N-vinylpyrrolidone); poly(vinylalcohol); polyethylene oxides; polyoxyethylene-polyoxypropylene copolymers (poloxamers); polysaccharides such as alginates; carrageenans; guar gum, karaya gum, gellan gum, agarose, locust bean gum, tragacanth gum, xanthan gum, and chitosan; hyaluronic acid; lecithin; and carbomer polymers (Carbopol®).

### I. Lubricating Agents

In one embodiment, the formulations of the invention may contain at least one lubricating agent. Lubricating agents useful in the compositions of the present technology include, but are not limited to, e.g., cellulose derivatives such as hydroxymethylcellulose, hydroxyethylcellulose, hydroxypropylcellulose, and hydroxypropylmethylcellulose.

### J. Cryopreservation Agents

Cryopreservation agent useful in the compositions of the present technology include, but are not limited to, e.g., carbohydrates including saccharides—disaccharides—and sugar alcohols, glycerol, polyalkoxyethers, PEG-fatty acids and lipids, biologically-based surfactants, and other surface active agents.

Cryopretectants useful in the compositions of the present technology include, but are not limited to, e.g., carbohydrates such as sucrose, xylose, glucose, and sugar alcohols such as mannitol and sorbitol, surface active agents such as the polysorbates (Tweens), as well as glycerol and dimethylsulfoxide. Cryoprotectants useful in the compositions of the present technology include, but are not limited to, e.g., water-soluble polymers such as polyvinylpyrrolidone (PVP), starch, and polyalkoxy ethers such as polyethylene glycols, polypropylene glycols, and poloxamers. Biologically derived cryoprotectants useful in the compositions of the present technology include, but are not limited to, e.g., albumin. Yet another class of cryoprotectant useful in the compositions of the present technology include, but are not limited to, e.g., PEGylated lipids, such as Solutol® HS 15 (polyethylene glycol 660 12-hydroxystearate).

### K. Non-ionic Surfactants

Nonionic surfactants useful in the compositions of the present technology include, but are not limited to, e.g., polyoxyethylene fatty alcohol ethers, polyoxyethylene sorbitan fatty acid esters, polyoxyethylene fatty acid esters, polyoxyethylene-derivatized lipids such as mPEG-PSPC (palmitoyl-stearoyl-phophatidylcholine), mPEG-PSPE (palmitoyl-stearoyl-phophatidylethanolamine), sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), polaxamines, methylcellulose, hydroxycellulose, hydroxypropylcellulose, hydroxypropyl methylcellulose, noncrystalline cellulose, polysaccharides, starch, starch derivatives, hydroxyethylstarch, polyvinyl alcohol, and polyvinylpyrrolidone.

### II. Method of Making the Charged Micelles of the Present Technology

The processes useful to prepare the charged micelles of the present technology are known to those skilled in the art. For instance, the manufacturing of drug-containing micelles may consist of the following processing steps: (1) addition and dissolution of a surfactant in purified water by low-shear mixing; (2) addition and dissolution of a drug in the surfactant solution from Step 1 by high-shear followed by low-shear mixing; (3) filtration and/or centrifugation of drug-containing micelle preparation from Step 2 to remove undissolved particulates from the solution; (4) addition and dissolution of inactive ingredients to the filtered micelle preparation from Step 3 by high-shear and/or low-shear mixing; (5) optionally lyophilization of the micelle preparation from Step 5; and (6) packaging and sterilization of the micelle preparation from Step 5 by one of the methods of heating, UV irradiation, or gamma irradiation.

### III. Methods of Using the Charged Micelles of the Present Technology

A wide variety of ocular conditions such as glaucoma, ocular inflammatory conditions such as keratitis, uveitis, intra-ocular inflammation, allergy and dry-eye syndrome ocular infections, ocular allergies, ocular infections (bacterial, fungal, and viral), cancerous growth, neo vessel growth originating from the cornea, retinal edema, macular edema, diabetic retinopathy, retinopathy of prematurity, degenerative diseases of the retina (macular degeneration, retinal dystrophies), and retinal diseases associated with glial proliferation may be prevented or treated using the charged micelle compositions according to the present technology.

In general, micelle compositions are prepared with consideration of drug physical and chemical properties, surfactant type, drug:surfactant molar ratio, method and route of delivery, and target therapeutic levels in ocular tissues.

### EXAMPLES

The following examples are intended to be non-limiting illustrations of certain embodiments of the present invention. All references cited are hereby incorporated herein by reference in their entireties.

### Example 1

### Solubilization of Sparingly Soluble Compounds using Quaternary Ammonium Cationic Surfactants

Aqueous solubility of dexamethsone, a corticosteroid, and natamycin, an antifungal agent, was increased by using quaternary ammonium cationic surfactants cetyltrimethylammonium bromide (CTAB), ditetradecyldimethylammonium bromide (TMAB), and dioctadecyldimethylammonium chloride (OMAC). Stock solutions of surfactants were prepared by adding water to the surfactant and mixing under low shear at room temperature. The stock solution of CTAB and TMAB was prepared at 10 mg/mL and 20 mg/mL in WFI (water for injection) whereas the stock solution of OMAC was only prepared at 10 mg/mL due to gel formation at 20 mg/mL. Surfactant solutions with different concentrations were then prepared by diluting the corresponding stock solution with WFI.

Dexamethasone (or natamycin) was added to each surfactant solution in such quantity to precipitate. The mixture was sonicated for one hour in a sonication water tank and then stirred overnight.

Dissolved dexamethasone (or natamycin) concentration was analyzed on HPLC following two different procedures: (1) An aliquot of the mixture was centrifuged at 10 k RPM (9.3 k×g relative gravitational force) for 30 min. Clear supernatant was then analyzed on HPLC; (2) An aliquot of the mixture was filtered through a syringe filter (Acrodisc®, Supor membrane, 0.2 micrometers), and then analyzed on HPLC.

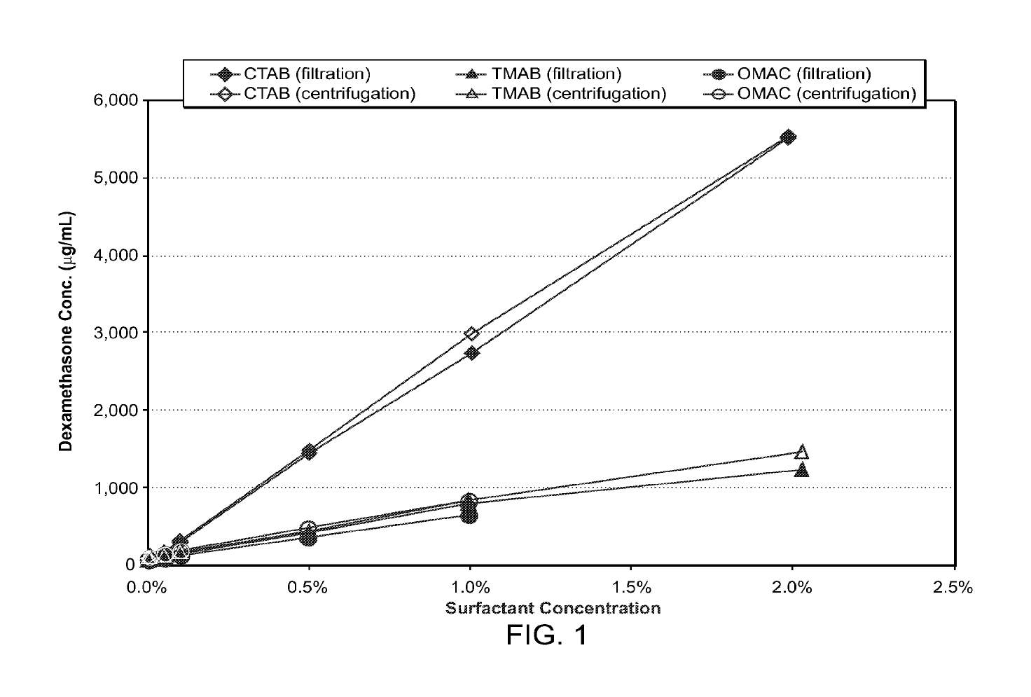

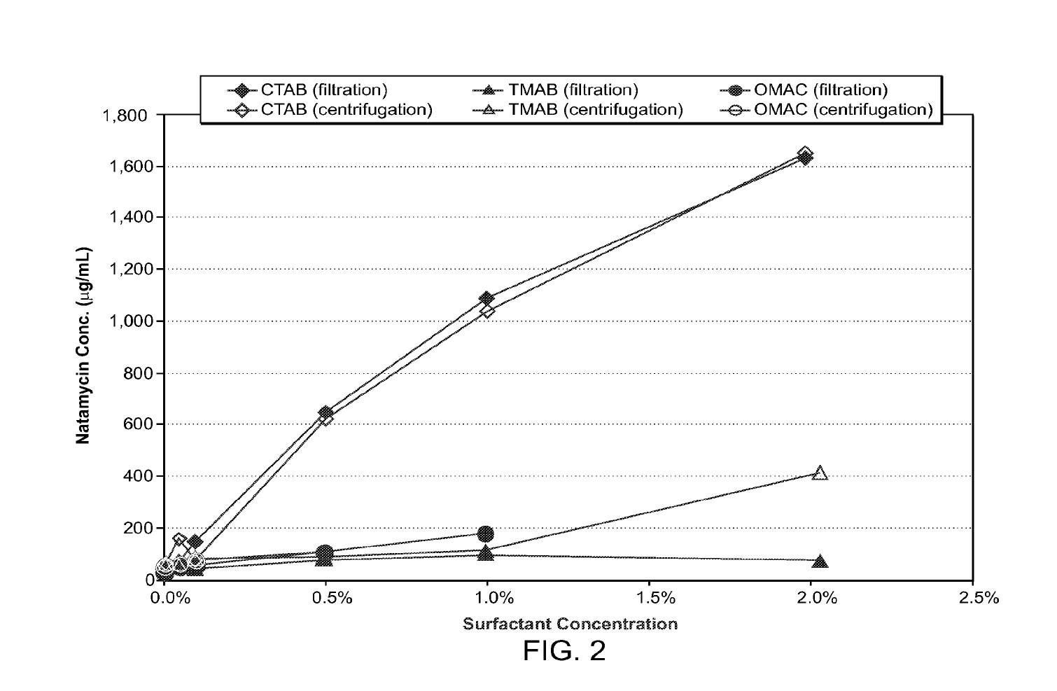

The dissolved concentrations of dexamethasone and natamycin vs. surfactant concentration are plotted in FIG. 1 and FIG. 2, respectively.

### EQUIVALENTS

Although the present technology has been described in considerable detail with reference to certain preferred versions thereof, other versions are possible. Therefore, the spirit and scope of the appended claims should not be limited to the description of the preferred versions contained herein. All references are herein incorporated by reference in their entireties.

What is claimed is:

1. A pharmaceutical composition suitable for ocular iontophoresis comprising a one or more populations of micelles comprising a one or more charged surfactant and a one or more bioactive agent.

2. A composition of claim 1 wherein the surface charge of micelles is characterized with a zeta potential ranging from about (+)20 mV to about (+)100 mV.

3. A composition of claim 1 wherein the surfactant is a quaternary ammonium cationic surfactant (QACS).

4. A composition of claim 3 wherein the QACS is selected from the group consisting of alkyltrimethylammonium halide, alkyldimethylammonium halide, alkylmethylammonium halide, alkylethyldimethylammonium halide, alkyldimethylbenzylammonium halide, alkylpyridinium halide, and alkylimidazolium halide, or a mixture of two or more thereof

5. A composition of claim 4 wherein the QACS is selected from the group consisting of decyltrimethylammonium halide, lauryltrimethylammonium halide, cetyltrimethylammonium halide, cetylethyldimethylammonium halide, octadecyltrimethylammonium halide, didodecyldimethylammonium halide, ditetradecyldimethylammonium halide, dioctadecyldimethylammonium halide, and trioctadecylmethylammonium halide, or a mixture of two or more thereof

6. A composition of claim 5 wherein the QACS is cetyltrimethylammonium bromide.

7. A composition of claim 5 wherein the QACS is didodecyldimethylammonium bromide.

8. A composition of claim 5 wherein the QACS is ditetradecyldimethylammonium bromide.

9. A composition of claim 5 wherein the QACS is dioctadecyldimethylammonium chloride.

10. The pharmaceutical composition of claim 1 further comprising at least one inactive ingredients selected from the group consisting of: buffering agents, osmotic agents, penetration or absorption enhancers, chelants, antioxidants, preservatives, pH adjusting agents, viscosity modifying agents, lubricating agents, non-ionic surfactants and cryopreservative agents.

11. A composition of claim 1 wherein the surface charge of micelles is characterized with a zeta potential ranging from about (−)20 mV to about (−)100 mV.

12. A method for preventing or treating an ocular disease or condition in a subject, the method comprising administering to a subject in which such treatment or prevention is desired an effective amount of a composition according to claim 1 by ocular iontophoresis.

13. The method of claim 12 wherein the method of delivery is transcorneal iontophoresis.

14. The method of claim 12 wherein the method of delivery is transscleral iontophoresis.

15. The method of claim 12, wherein the ocular disease or condition is selected from the group consisting of: glaucoma, ocular inflammatory conditions such as keratitis, uveitis, intra-ocular inflammation, allergy and dry-eye syndrome ocular infections, ocular allergies, ocular infections (bacterial, fungal, and viral), cancerous growth, neo vessel growth originating from the cornea, retinal oedema, macular oedema, diabetic retinopathy, retinopathy of prematurity, degenerative diseases of the retina (macular degeneration, retinal dystrophies), and retinal diseases associated with glial proliferation.
